(19) United States
(12) Patent Application Publication
(10) Pub. No.: US 2020/0342992 A1
(43) Pub. Date: Oct. 29, 2020

(54) HEALTH MONITORING SYSTEMS AND METHODS HAVING PERSONAL HEALTH ASSESSMENT DEVICES

(71) Applicant: THE BOEING COMPANY, Chicago, IL (US)

(72) Inventors: Cynthia A. Vandewall, Snohomish, WA (US); Elizabeth A. O'Hearn, Cincinnati, OH (US); Brian Keller, Cincinnati, OH (US); Blake Lane, Cincinnati, OH (US); Craig M. Vogel, Cincinnati, OH (US); Shuai Mu, Datong (CN); Chuyu Ruan, GuangZho (CN); Swati D. Chopra, Bevercreek, OH (US)

(73) Assignee: THE BOEING COMPANY, CHICAGO, IL (US)

(21) Appl. No.: 16/393,269

(22) Filed: Apr. 24, 2019

(51) Int. Cl. G16H 50/30; G06F 9/451

(52) U.S. Cl. CPC G16H 50/30; G06F 9/451

(57) ABSTRACT

A health monitoring system and method configured to monitor health of one or more individuals within an internal cabin of a vehicle include one or more personal health assessment devices associated with the one or more individuals. The personal health assessment device(s) obtain health data from the individual(s) and output health signals including the health data to one or more group health monitoring devices associated with one or more attendants who are responsible for taking care of the individual(s).

### FIELD OF THE DISCLOSURE

Embodiments of the present disclosure generally relate to health monitoring systems and methods, such as may be used onboard a commercial aircraft during a flight.

### BACKGROUND OF THE DISCLOSURE

Various types of aircraft are used to transport passengers between various locations. During a flight, attendants take care of the passengers. For example, the passengers may order drinks, which are delivered to the passengers at their seats. Further, the attendants may deliver various food items to the passengers. In general, the attendants typically ensure that the passengers are comfortable during the flight.

For various reasons, passengers may contact an attendant by engaging a call button, which is typically positioned above a seat of the passenger. A passenger may engage the call button to order a drink, or to inform the attendant of discomfort, such as if the passenger is feeling ill.

Many individuals are flying more nowadays. As an example, many business travelers fly frequently. Indeed, some business travelers fly multiple times in a month, if not a week. Moreover, many travel plans include multiple flights. As an example, a traveler flying from Chicago, Ill. to Sydney, Australia may arrive at and depart from multiple airports before arriving at the final destination. As can be appreciated, the time onboard planes for certain passengers in a given travel day may last multiple hours.

Many passengers who fly frequently are increasingly savvy about maintaining their health and wellness, especially when traveling, due to having important business within a relatively short time after landing at their destination. As such, the passengers prefer to be feeling well, alert, and in a best possible condition upon arrival at the destination.

However, various passengers may not pay attention to certain health aspects during a flight. For example, certain passengers may be so focused on other topics (such as preparing for a work meeting at the destination) that they do not realize their hydration levels are low. As another example, certain passengers may fly infrequently, and may not be aware of certain services available and/or less in tune with certain health aspects during a flight. With long haul flights, in particular, certain passengers may become dehydrated if they refrain from drinking water for too long. Further, during such flights, certain passengers may also not realize they are fatigued. Nevertheless, attendants are often able to alleviate passenger discomfort by providing water, a pillow, a blanket, or the like. However, an attendant typically has no way of knowing that a passenger is thirsty or tired unless the passenger informs the attendant.

Moreover, certain passengers may be reluctant to use a call button during a flight. When a call button is engaged, a visual indication proximate the seat of the passenger is noticeable. As such, certain passengers may opt not to use the call button due to embarrassment. Further, certain passengers may not want to bother or otherwise burden an attendant, who they know are responsible for taking care of numerous passengers onboard the aircraft. Nevertheless, a passenger may truly be in need of attention (such as for medical reasons), but reluctant to inform an attendant, who otherwise has no way of knowing that the passenger is in need of attention.

### SUMMARY OF THE DISCLOSURE

A need exists for a system and a method for attending to a group of individuals, such as onboard an aircraft. Further, a need exists for a system and a method for attending to the group of individuals that need not rely on the individuals themselves to contact attendants for service and assistance. Moreover, a need exists for a system and a method for attending to a group of individuals onboard an aircraft that does not require a passenger to engage a physical call button.

With those needs in mind, certain embodiments of the present disclosure provide a health monitoring system configured to monitor health of one or more individuals within an internal cabin of a vehicle. The health monitoring system includes one or more personal health assessment devices associated with the individual(s). The more personal health assessment device(s) obtain health data from the individual(s) and output health signals including the health data to one or more group health monitoring devices associated with one or more attendants who are responsible for taking care of the individual(s).

In at least one embodiment, a plurality of personal health assessment devices is associated with a plurality of the individuals. Each of the plurality of personal health assessment devices is associated with a respective one of the plurality of the individuals.

The personal health assessment device(s) may be associated with one or more seats within the internal cabin.

In at least one embodiment, the personal health assessment device(s) include a health assessment control unit that obtains the health data. Further, the personal health assessment device(s) may include a user interface in communication with the health assessment control unit. The health data is input into the health assessment control unit via an input device of the user interface.

In at least one embodiment, the user interface includes a virtual call button.

In at least one embodiment, the personal health assessment device(s) include an imaging device in communication with the health assessment control unit. The imaging device captures image data of the individual(s) associated with the personal health assessment device(s). The health assessment control unit analyzes the image data to determine the health data.

The personal health assessment device(s) may also include one or more health monitoring devices in communication with the health assessment control unit. The health monitoring device(s) obtain the health data.

The health assessment control unit may include an interaction module that displays messages on a screen and allows the individual(s) to input the health data. The health assessment control unit may include a hydration assessment module that analyzes the images to determine a hydration level of the individual(s), a fatigue assessment module that analyzes the images to determine a fatigue level of the individual(s), a heart rate assessment module that analyzes the images to determine a heart rate of the individual(s), and/or an oxygen level assessment module that analyzes the images to determine a blood oxygen level of the individual(s).

In at least one embodiment, the health data relates to one or more of hydration level, fatigue level, heart rate, blood pressure, and/or blood oxygen level.

In at least one embodiment, at least one of the personal health assessment devices includes a mirror having a reflective surface, an imaging device that captures an image of the one or more individuals through the reflective surface, and a screen that shows graphics and text on the reflective surface.

Certain embodiments of the present disclosure provide a health monitoring method configured to monitor health of one or more individuals within an internal cabin of a vehicle. The health monitoring method includes associating one or more personal health assessment devices with the individual(s), obtaining, by the one or more personal health assessment devices, health data from the individual(s), and outputting, by the personal health assessment device(s), health signals including the health data to one or more group health monitoring devices associated with one or more attendants who are responsible for taking care of the individual(s).

### BRIEF DESCRIPTION OF THE DRAWINGS

FIG. 1 illustrates a schematic box diagram of a health monitoring system within an internal cabin of a vehicle, according to an embodiment of the present disclosure.

FIG. 2 illustrates a schematic box diagram of a personal health assessment device, according to an embodiment of the present disclosure.

FIG. 3 illustrates a schematic box diagram of a health assessment control unit, according to an embodiment of the present disclosure.

FIG. 4 illustrates a front view of the personal health assessment device, according to an embodiment of the present disclosure.

FIG. 5 illustrates a front view of the personal health assessment device, according to an embodiment of the present disclosure.

FIG. 6 illustrates a flow chart of a method of outputting a health signal including health data of an individual, according to an embodiment of the present disclosure.

FIG. 7 illustrates a front view of a user interface of the personal health assessment device showing health data regarding blood pressure and heart rate, according to an embodiment of the present disclosure.

FIG. 8 illustrates a front view of a user interface of the personal health assessment device showing health data regarding blood oxygen level, according to an embodiment of the present disclosure.

FIG. 9 illustrates a front view of a user interface of the personal health assessment device showing an image of an individual associated with the personal health assessment device, according to an embodiment of the present disclosure.

FIG. 10 illustrates a front view of a user interface of the personal health assessment device showing health data regarding hydration level, according to an embodiment of the present disclosure.

FIG. 11 illustrates a schematic box diagram of a group health monitoring device, according to an embodiment of the present disclosure.

FIG. 12 illustrates a front view of a user interface of the group health monitoring device, according to an embodiment of the present disclosure.

FIG. 13 illustrates a flow chart of a method of displaying health status of individuals associated with personal health assessment devices, according to an embodiment of the present disclosure.

FIG. 14 illustrates a flow chart of a method of predicting inventory for future routes of vehicles, according to an embodiment of the present disclosure.

FIG. 15 illustrates a schematic diagram of a personal health assessment device, according to an embodiment of the present disclosure.

FIG. 16 illustrates a perspective internal view of a lavatory, according to an example of the present disclosure.

FIG. 17 illustrates a front perspective view of an aircraft, according to an exemplary embodiment of the present disclosure.

### DETAILED DESCRIPTION OF THE DISCLOSURE

The foregoing summary, as well as the following detailed description of certain embodiments, will be better understood when read in conjunction with the appended drawings. As used herein, an element or step recited in the singular and preceded by the word “a” or “an” should be understood as not necessarily excluding the plural of the elements or steps. Further, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular condition may include additional elements not having that condition.

Certain embodiments of the present disclosure provide health monitoring systems and methods that monitor health and wellness of individuals within an area (such as an internal cabin of an aircraft). The health monitoring systems and methods include one or more control units that assess various health aspects of individuals, such as hydration, fatigue, oxygen levels, heart rate, and the like. The health aspects are received by one or more group health monitoring devices, which are reviewed by one or more attendants of the group. In this manner, the attendant(s) are able to monitor the group to determine which individuals are in need of assistance.

Certain embodiments of the present disclosure provide health monitoring systems and methods that monitor and manage the health of a passenger onboard an aircraft. In at least one embodiment, a group health monitoring device receives health data, which may be input from the passenger via a question and answer process, or directly via hardware such as a camera. For example, a camera on a personal health assessment device may be used to detect one or more health signals, such as heart rate, blood oxygen level, hydration, fatigue, or the like. In at least one embodiment, information such as travel schedule, flight profile information, or even weather forecasts and the like may be received, which allows the systems and methods to provide suggestions to the passenger for ensuring comfort. In at least one embodiment, a post trip survey may be employed to gather actual results data for comparison and customer satisfaction purposes. Over time, the aforementioned data may be collected and saved in order to provide an even better model and better suggestions for ensuring passenger comfort.

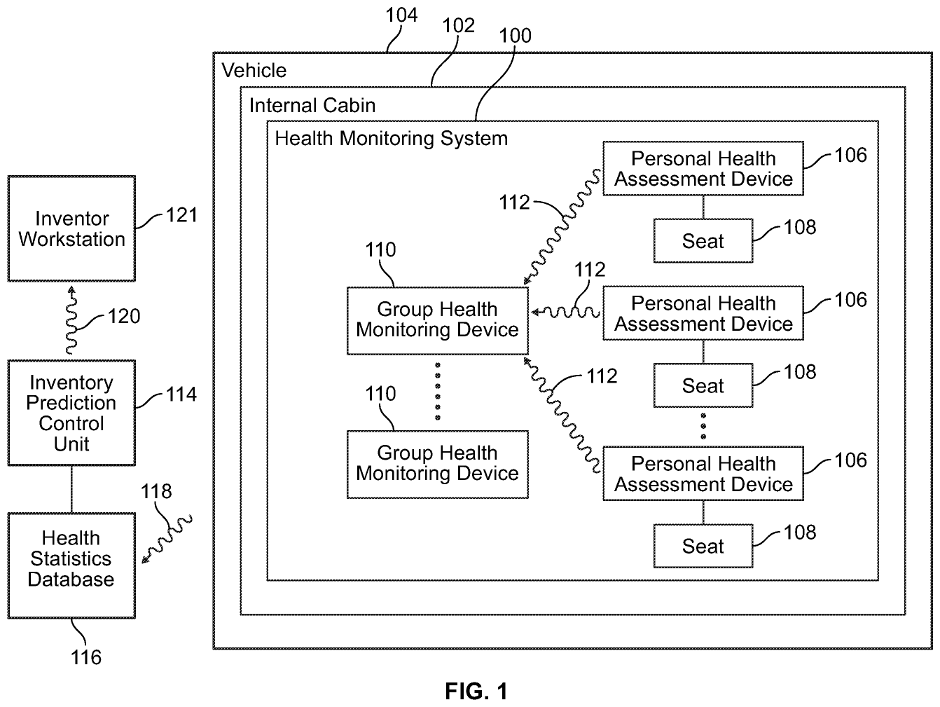

FIG. 1 illustrates a schematic box diagram of a health monitoring system 100 within an internal cabin 102 of a vehicle 104, according to an embodiment of the present disclosure. In at least one embodiment, the vehicle 104 is a commercial aircraft. In at least one other embodiment, the vehicle 104 is a train, a bus, a seacraft, or a spacecraft. The internal cabin 102 of the vehicle 104 is an example of an area in which the health monitoring system 100 is used. In at least one other embodiment, the health monitoring system 100 may be used in non-vehicle settings. For example, in at least one embodiment, the health monitoring system 100 is used in an area, such as a classroom, a theater, a restaurant, or the like. That is, the health monitoring system 100 is not limited to use with vehicles.

The health monitoring system 100 includes a plurality of personal health assessment devices 106. The personal health assessment devices 106 are associated with individuals, such as passengers, within the internal cabin 102. For example, each personal health assessment device 106 is associated with a particular seat 108 within the internal cabin 102. Optionally, the personal health assessment devices 106 need not be associated with seats 108.

The personal health assessment devices 106 are communicatively coupled to one or more group health monitoring devices 110 within the internal cabin 102, such as through one or more wireless connections (for example, Bluetooth connections). Optionally, at least some of the personal health assessment devices 106 may be in communication with the group health monitoring devices 110 through one or more wired connections. The group health monitoring devices 110 are associated with attendants who are responsible for taking care of the individuals associated with the personal health assessment devices 106. For example, each flight attendant onboard an aircraft may be assigned a group health monitoring device 110, which is in communication with the personal health assessment devices 106 associated with passengers onboard the aircraft.

In operation, the personal health assessment devices 106 obtain health data (that is, individual health data, in contrast to group health data) from the passengers. The personal health assessment devices 106 output health signals 112, which are received by the group health monitoring devices 110. Attendants are then able to monitor the health status of the individuals associated with the personal health assessment devices 106 via the group health monitoring devices 110. As such, the attendants are able to objectively determine which passengers may be in need of service, assistance, or other such attention without the need for the passengers actively contacting the attendants.

In at least one embodiment, the health signals 112 including the health data of the individuals that are received by the group health monitoring devices 110 may not include personal identification information (such as a name, social security or other such number, or the like) of the individuals associated with the personal health assessment devices 106. Instead, the health signals 112 may include the location of the seats 108, or other generic information that identifies a location of the individual within the internal cabin 102. In at least one embodiment, certain aspects of the individuals may be contained within the health data that is on the health signals 112, such as age, sex, certain health conditions, or the like. In at least one embodiment, the health data may only be stored after an individual provides express authorization for storage of such health information. In at least one embodiment, the health data may only be stored on a personal health assessment device 106 that is owned by an individual (that is, the health data is not stored by a third party). In at least one embodiment, the health data may be stored during travel for an individual from a departure location to an arrival location, whether the travel includes only one leg (for example, one flight) or multiple legs (for example, two or more flights). The health data may be deleted after such travel, and the individual may receive a message indicating that such health data has been deleted. In at least one embodiment, the individual may be prompted to delete such health data.

In at least one embodiment, the health monitoring system 100 also includes an inventory prediction control unit 114 that is in communication with a health statistics database 116, such as through one or more wired or wireless connections. As shown, the inventory prediction control unit 114 and the health statistics database 116 are remotely located from the vehicle 104. In at least one other embodiment, one or both of the inventory prediction control unit 114 or the health statistics database 116 may be onboard the vehicle 104.

In operation, the health statistics database 116 receives group health signals 118 that include group health data from the group health monitoring device(s) 110. For example, at the completion of a flight, the group health signals 118 may be transmitted to the health statistics database 116. The group health signals 118 include the health data (as received by the group health monitoring devices 110 from the personal health assessment devices 106) for the individuals associated with the personal health assessment devices 106 during the flight. The health statistics database 116 associates the general information for the individuals (for example, not including personal identification information) with health data during the flight. As an example, the group health data may indicate that a certain number of passengers were dehydrated during the flight.

The inventory prediction control unit 114 analyzes the group health data for prior travel, such as a prior flight, to predict inventory for a future travel, such as a future flight. For example, the inventory prediction control unit 114 analyzes the group health data for the prior flight. Based on the group health data for the prior flight, the inventory prediction control unit determines an amount of bottled water, pillows, blankets, and other such supplies that should be provided for future travel. In at least one embodiment, the inventory prediction control unit 114 determines that a certain amount of bottled water will be requested by the passengers of a future flight, based on the number of passengers who experienced at least some degree of dehydration or under-hydration during the prior flight. Similarly, the inventory prediction control unit 114 determines that a certain number of the passengers of a future flight will request a pillow and/or blanket based on the number of passengers who experienced some degree of fatigue during the prior flight. The inventory prediction control unit 114 uses such information and statistical models, which may be programmed in memory, to determine future inventory (such as requests for bottled water, pillows, blankets, and the like), and outputs an inventory prediction signal 120, which includes inventory prediction data. The inventory prediction signal 120 is received at an inventory workstation 121 (such as a computer workstation, handheld device, or the like), where inventory may be ordered for future flights.

In at least one embodiment, the health statistics database 116 stores group health signals 118 for numerous events over a selected time frame. For example, the health statistics database 116 stores group health signals 118 for route (for example, flights) of one or more vehicles (for example, aircraft) for a week, month, year, or longer. In at least one embodiment, the group health signals 118 are associated with particular flights, dates of flights, and the like. As such, the inventory prediction control unit 114 is able to predict inventory for various flights at different times of year. For example, the inventory prediction control unit 114 analyzes the group health data as received by the group health signals 118 for flights at different times of year to determine an inventory prediction that may differ due to the different times of year.

Again, the group health data may be devoid of information that could specifically identify the individuals. Instead, the group health data may include health data for the group as a whole (for example, 5 passengers on flight A were dehydrated, 3 passengers on flight B had elevated heart rates during the flight, etc.).

Alternatively, the health monitoring system 100 may not include the inventory prediction control unit 114 and/or the health statistics database 116.

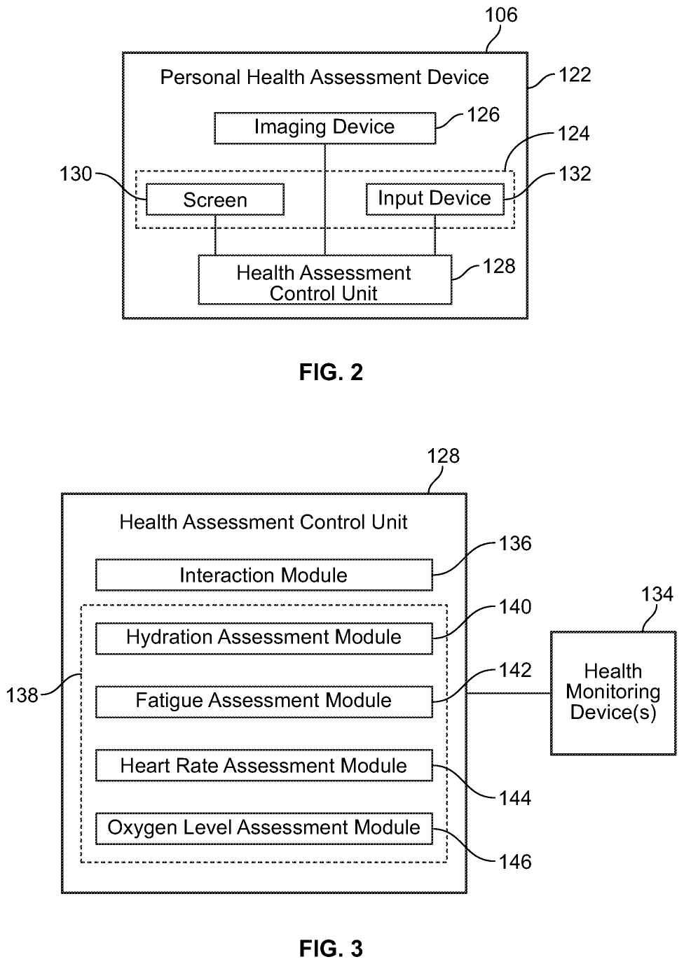

FIG. 2 illustrates a schematic box diagram of a personal health assessment device 106, according to an embodiment of the present disclosure. In at least one embodiment, the personal health assessment device 106 includes a housing 122 that contains a user interface 124, an imaging device 126, and a health assessment control unit 128.

The user interface 124 may include a screen 130 and an input device 132, which are in communication with the health assessment control unit 128, such as through one or more wired or wireless connections. The screen 130 may be a computer or television monitor, a digital display, or the like. The input device 132 may be a keyboard, stylus, mouse, or the like. In at least one embodiment, the screen 130 and the input device 132 are integrated together as a touchscreen interface. In at least one embodiment, the imaging device 126 is a camera, such as an electronic photographic or video camera, that is configured to capture image data of an individual associated with the personal health assessment device 106.

The health data relates to one or more of hydration level, fatigue level, heart rate, blood pressure, blood oxygen level, and/or the like. In at least one embodiment, the individual inputs health data into the personal health assessment device 106 via the input device 132. For example, the individual may input a blood pressure, heart rate, hydration level, or the like via the input device 132. The health assessment control unit 128 analyzes the input data and outputs such data as the health signal 112 (shown in FIG. 1).

In at least one embodiment, the health data is determined by the health assessment control unit 128 analyzing one or more captured images (including photographic or video images) of an individual obtained via the imaging device 126. For example, the imaging device 126 obtains images of the individual, which are then received by the health assessment control unit 128. The health assessment control unit 128 analyzes the images of the individual to determine the health data from image recognition techniques. For example, the health assessment control unit 128 may determine a hydration level, a fatigue level, a blood oxygen level, a glucose level, and/or the like through analysis of the images of the individual's face (or features thereof), or other anatomy, such as a neck, wrist, finger, or the like. The health assessment control unit 128 determines the health data from the images and outputs such data as the health signal 112 (shown in FIG. 1).

FIG. 3 illustrates a schematic box diagram of the health assessment control unit 128, according to an embodiment of the present disclosure. As noted, the health assessment control unit 128 is configured to determine health data of the individual through one or more of data input by the individual, recognition of biometric aspects within one or more images of the individual, and/or signals received from one or more health monitoring devices 134 that may be in communication with the health assessment control unit 128, such as through one or more wired or wireless signals.

Referring to FIGS. 2 and 3, in at least one embodiment, the health assessment control unit 128 receives the health data as input by the individual through the user interface 124, such as the input device 132. In one example, the health assessment control unit 128 includes an interaction module 136 (such as one or more processors, circuits, and/or the like) that displays messages on the screen 130 and allows the individual to input the health data into the personal health assessment device 106.

In at least one embodiment, the health assessment control unit 128 determines the health data by analyzing one or images of the individual, as captured by the imaging device. As an example, the health assessment control unit 128 determines one or more of a hydration level, a fatigue level, a heart rate, an oxygen level, or the like of the individual through an analysis of the image(s) of the individual. In at least one embodiment, the health assessment control unit 128 may include one or more health aspect assessment modules 138 (such as one or more processors, circuits, and/or the like) that analyze the images to determine the health data. For example, the health aspect assessment modules 138 include a hydration assessment module 140 that analyzes the image(s) to determine a hydration level of the individual, a fatigue assessment module 142 that analyzes the image(s) to determine a fatigue level of the individual, a heart rate assessment module 144 that analyzes the image(s) to determine a heart rate of the individual, and an oxygen level assessment module 146 that analyzes the images to determine a blood oxygen level of the individual. The health aspect assessment modules 138 may include more or less modules than shown. For example, the health aspect assessment modules 138 may include a glucose assessment module to determine a blood glucose level of the individual.

The health monitoring devices 134 may be separate and distinct devices that communicatively couple to the personal health assessment device 106 to detect various health aspects of the individual. The health monitoring devices 134 may be separate devices that couple to the personal health assessment device, such as may be part of a seat (for example, a heart rate monitor embedded within a passenger seat), a separate attachment device (such as a pulse oximetry probe that removably connects to the personal health assessment device 106), a monitor (such as a heart rate monitor, wristband, smart watch, etc.) worn by the individual, or the like. Examples of the health monitoring devices 134 include heart rate monitors (such as may be included with smart watches, bands, devices, or the like), hydration level monitors (such as may be positioned on a finger of the individual), a pulse oximetry probe (such as may be positioned on a finger of the individual), or the like. The health aspect assessment modules 138 may receive the health data from the health monitoring devices 134 in addition to, or instead of, determining the health data through analysis of one or more images of the individual.

The health assessment control unit 128 assesses health data of the individual as obtained from (a) the individual inputting the health information via the user interface 124, (b) analysis of one or more images of the individual as captured by the imaging device 126, and/or (c) the one or more health monitoring devices 134. The health assessment control unit 128 obtains the health data and outputs the health signal 112, which includes the health data, to the group health monitoring device 110, as shown in FIG. 1.

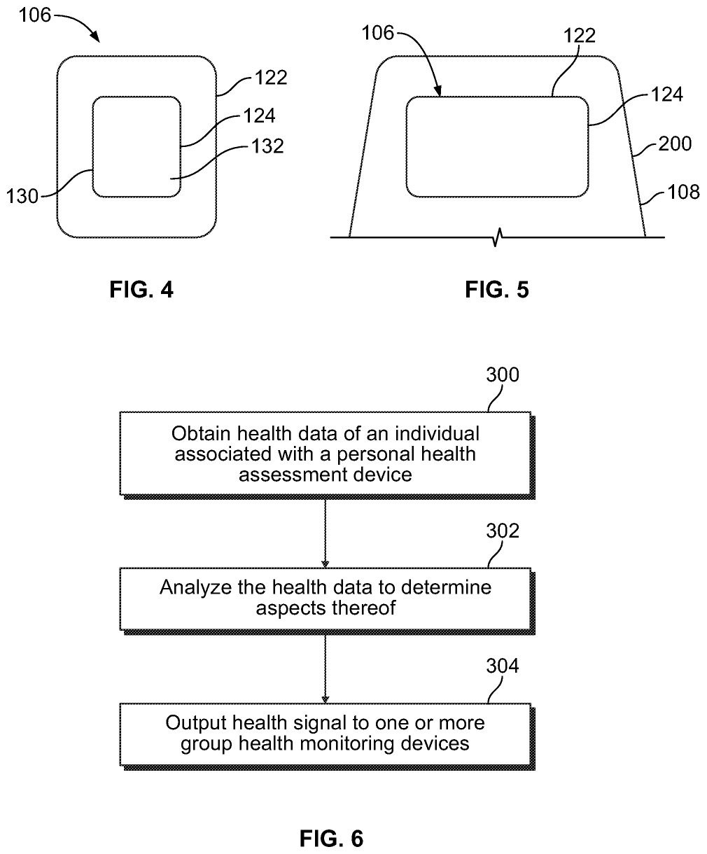

FIG. 4 illustrates a front view of the personal health assessment device 106, according to an embodiment of the present disclosure. In at least one embodiment, the personal health assessment device 106 is a handheld smart device, such as a smart phone or tablet. The user interface 124 may be a touchscreen interface that includes the screen 130 and the input device 132. For example, the personal health assessment device 106 may be, or part of, a handheld device of an individual.

FIG. 5 illustrates a front view of the personal health assessment device 106, according to an embodiment of the present disclosure. In at least one embodiment, the personal health assessment device 106 may be mounted to, formed with, or otherwise disposed on a rear of headrest 200 of a seat 108 in front of a seat of individual associated with the personal health assessment device 106. In at least one other embodiment, the personal health assessment device 106 may be on the seat for the individual associated with the personal health assessment device 106, such as an armrest of the seat. In at least one other embodiment, the personal health assessment device 106 may be part of a screen that extends from a ceiling above a seat.

FIG. 6 illustrates a flow chart of a method of outputting a health signal including health data of an individual, according to an embodiment of the present disclosure. In at least one embodiment, the personal health assessment device 106 shown in FIGS. 1-5 operates according to the flow chart of FIG. 6.

Referring to FIGS. 1-6, at 300, health data is obtained by the health assessment control unit 128 of the personal health assessment device 106. The health data may be obtained through the individual inputting the health data into the personal health assessment device, analysis of images captured by the imaging device 126, and/or as detected by the health monitoring device(s) 134. At 302, the health assessment control unit 128 analyzes the health data to determine aspects thereof, such as levels of hydration and fatigue (such as by comparing the hydration levels or fatigue levels of the individual to stored thresholds related to degrees of hydration and fatigue). Then, at 304, the health assessment control unit 128 outputs the health signal 112, which includes the health data including aspects thereof). The health signal is received by one or more of the group health monitoring devices 110.

As noted, in at least one embodiment, the health assessment control unit 128 compares the health data to predetermined thresholds stored in memory to determine degrees of hydration and fatigue. For example, the health assessment control unit 128 determines that a hydration level of an individual above a first hydration level is adequate or normal hydration. However, the health assessment control unit 128 determines that a hydration level of an individual below the first hydration level, but above a second hydration level that is lower than the first hydration level is mild dehydration. Further, the health assessment control unit 128 determines that a hydration level of an individual below the second hydration level is severe dehydration. The stored dehydration levels for determining hydration levels are predetermined, such as by medical boards, research, and findings, for example. More or less thresholds may be used.

Similarly, the health assessment control unit 128 determines that a fatigue level of an individual above a first fatigue level is non-fatigued. Further, the health assessment control unit 128 determines that a fatigue level of an individual below the first fatigue level, but above a second fatigue level that is lower than the first fatigue level is mild fatigue. Also, the health assessment control unit 128 determines that a fatigue level of an individual below the second fatigue level is severe fatigue. The stored fatigue levels are predetermined, such as by medical boards, research, and findings, for example. More or less thresholds may be used. Similarly, normal and elevated thresholds that may be stored for heart rate, blood pressure, glucose level, blood oxygen level, and the like are predetermined, such as by commonly accepted medical research, findings, and the like.

Referring to FIGS. 1-6, in at least one embodiment, a health monitoring method is configured to monitor health of one or more individuals within the internal cabin 102 of the vehicle 104. The health monitoring method includes associating one or more of the personal health assessment devices 106 with the individual(s), obtaining, by the personal health assessment device(s) 106, health data from the individual(s), and outputting, by the personal health assessment device(s) 106, health signals 112 including the health data to one or more group health monitoring devices 110 associated with one or more attendants who are responsible for taking care of the individual(s).

In at least one embodiment, the associating includes associating a plurality of the personal health assessment devices 106 with a plurality of the individuals. Each of the plurality of personal health assessment devices 106 is associated with a respective one of the plurality of the individuals. In at least one embodiment, the associating includes associating the personal health assessment device(s) 106 with one or more of the seats 108 within the internal cabin 102.

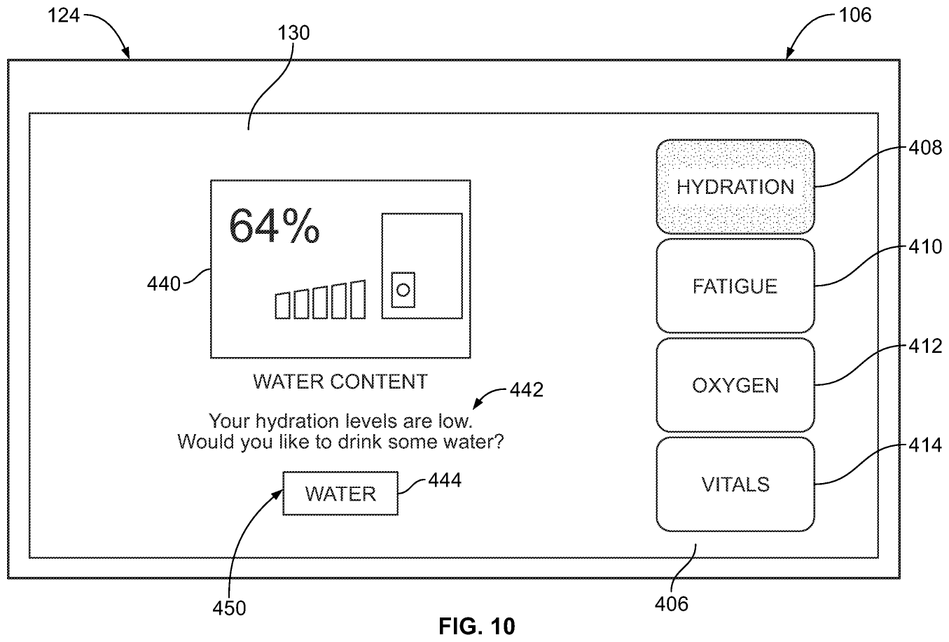

In at least one embodiment, the obtaining includes obtaining the health data with a health assessment control unit 128 of the personal health assessment device(s) 106. The obtaining may include inputting the health data into the health assessment control unit 128 via the input device 132 of the user interface 124. The method may also include providing a virtual call button (such as the virtual call button 450 shown in FIG. 10) on the user interface 124.

The obtaining may include capturing image data of the individual(s) by the imaging device 126, and analyzing the image data by the health assessment control unit 128 to determine the health data. The obtaining may include obtaining the health data by one or more health monitoring devices 134 in communication with the health assessment control unit 128.

In at least one embodiment, the method includes displaying, by the interaction module 136 of the health assessment control unit 128, messages on the screen 130 to allow the individual(s) to input the health data.

In at least one embodiment, the obtaining includes analyzing, by one or more of the health aspect assessment modules 138 of the health assessment control unit 128, images of the individuals to determine the health data. For example, the analyzing may include analyzing, by the hydration assessment module 140, the images to determine a hydration level of the individuals, analyzing, by the fatigue assessment module 142, the images to determine a fatigue level of the individual(s), analyzing, by the heart rate assessment module 144, the images to determine a heart rate of the individual(s), and/or analyzing, by the oxygen level assessment module 146, the images to determine a blood oxygen level of the individual(s).

In at least one embodiment, the health monitoring method also includes showing the health data of the individual(s) on the group health monitoring device(s) 110.

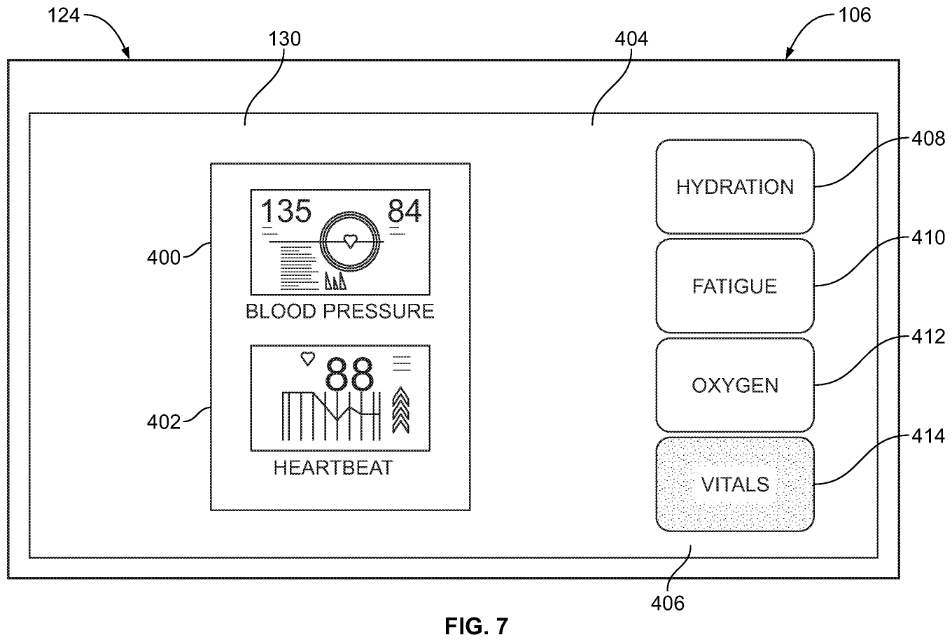

FIG. 7 illustrates a front view of the user interface 124 of the personal health assessment device 106 showing health data regarding blood pressure 400 and heart rate 402, according to an embodiment of the present disclosure. As shown, the user interface 124 includes a touchscreen interface that allows the individual to interact with the personal health assessment device 106 through touching the screen 130. The screen 130 includes a health data area 404 that shows the obtained health data, such as the current blood pressure 400 and the current heart rate 402 of the individual associated with the personal health assessment device 106. The screen 130 also includes a selection area 406 that allows the individual to select display of different health data, including hydration 408, fatigue 410, blood oxygen 412, and vitals 414 (including the blood pressure 400 and the heart rate 402). In response to the individual selecting the vitals 414 in the selection area, the health assessment control unit 128 displays the current vitals of the individual on the screen 130.

The selection area 406 may include options for more or less health data than shown. In at least one embodiment, the health data shown on the personal health assessment device 106 is received by the group health monitoring device(s) 110 (shown in FIG. 1).

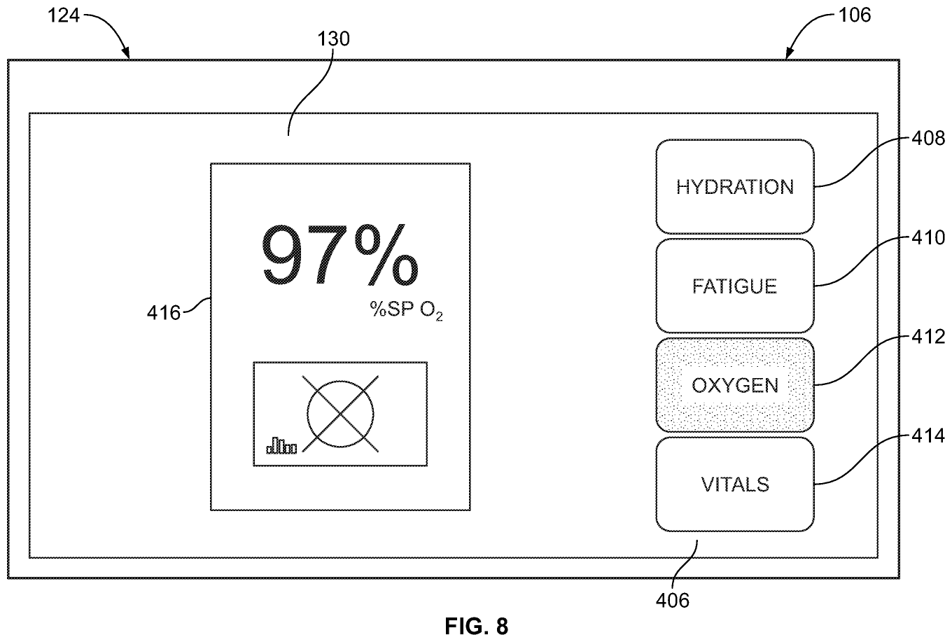

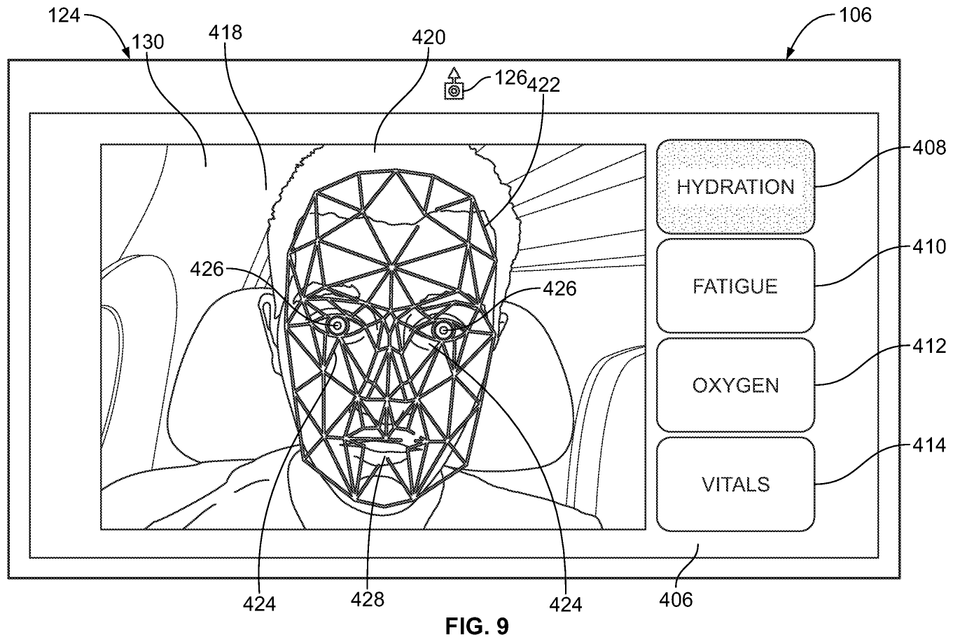

FIG. 8 illustrates a front view of the user interface 124 of the personal health assessment device 106 showing health data regarding blood oxygen level 416, according to an embodiment of the present disclosure. In response to the individual selecting the blood oxygen 412 in the selection area 406, the health assessment control unit 128 displays the current blood oxygen level 416 of the individual on the screen 130.

FIG. 9 illustrates a front view of the user interface 124 of the personal health assessment device 106 showing an image 418 of an individual 420 associated with the personal health assessment device 106 according to an embodiment of the present disclosure. The image 418 is captured by the imaging device 126, and includes one or more biometric identifiers 422 that are analyzed by the health assessment control unit 128 to determine health data of the individual 420. For example, the health assessment control unit 128 may determine health data such as hydration levels, fatigue, blood oxygen levels, heart rate, blood pressure, and/or the like via features of the biometric identifiers 422, such as skin color variation (for example, shades of red indicating blood flow), coloration 424 below and/or within the eyes 426, coloration of lips 428, and/or the like.

FIG. 10 illustrates a front view of the user interface 124 of the personal health assessment device 106 showing health data regarding hydration level 440, according to an embodiment of the present disclosure. In response to the individual selecting the hydration 408 in the selection area 406, the health assessment control unit 128 displays the current hydration level 440 of the individual on the screen 130. The health assessment control unit 128 may determine that the hydration level is low, and may display a message 442 on the screen indicating as much, and offering an option to contact an attendant for water via a water indicator 444. In this manner, the attendant may be alerted to the low hydration level of the individual and bring the individual a bottle of water, such as if requested by the individual via contacting the water indicator 444.

The water indicator 444 is an example of a virtual call button 450 that allows the individual to discreetly contact the attendant for service. Engagement of the virtual call button 450 may not illuminate a light proximate to the individual's seat, or otherwise indicate to other passenger's that the individual has requested attention from the attendant. Further, the virtual call button 450 may replace a physical call button (and associated wiring) proximate to the seat, thereby reducing weight of an aircraft, for example.

In at least one embodiment, information such as travel schedule, flight profile information, or weather forecasts and the like may be received by the personal health assessment devices 106. For example, a travel schedule and/or flight profile information may be input by an individual into the personal health assessment device 106. In at least one other embodiment, the travel schedule and/or the flight profile information may be transmitted to the personal health assessment device 106, such as by a travel provider. Further, weather forecasts may be received from weather forecast services, the Internet, or the like. The health assessment control unit 128 may analyze such information, and provide comfort suggestions to the individual associated with the personal health assessment device 106. For example, the health assessment control unit 128 may display messages on the screen 130 regarding suggested hydration levels in anticipation of the weather at a particular destination. In at least one embodiment, the health assessment control unit may provide a post trip survey on the screen 130, which may be used to gather actual results data for comparison and customer satisfaction purposes.

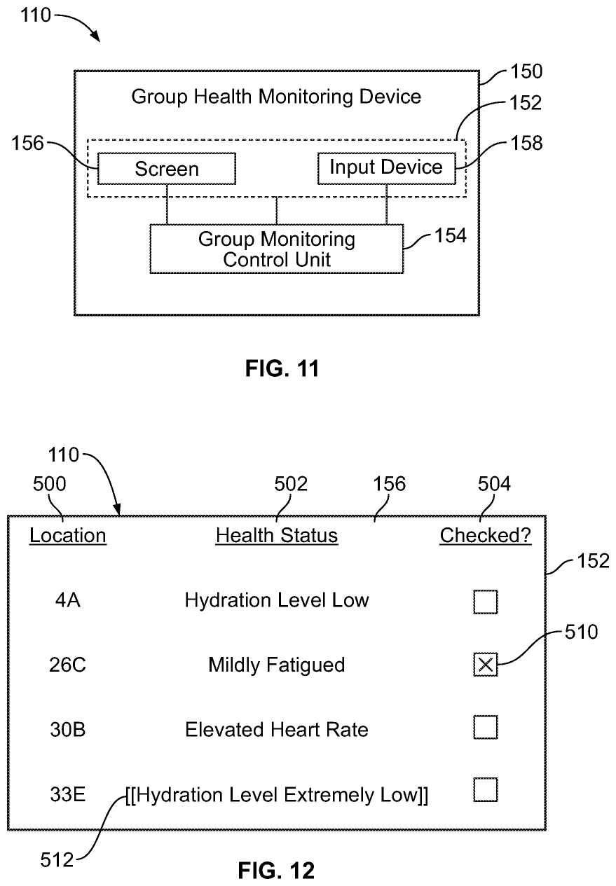

FIG. 11 illustrates a schematic box diagram of a group health monitoring device 110, according to an embodiment of the present disclosure. In at least one embodiment, the group health monitoring device 110 includes a housing 150 that contains a user interface 152 in communication with a group monitoring control unit 154.

The user interface 152 may include a screen 156 and an input device 158, which are in communication with the group monitoring control unit 154, such as through one or more wired or wireless connections. The screen 156 may be a computer or television monitor, a digital display, or the like. The input device 158 may be a keyboard, stylus, mouse, or the like. In at least one embodiment, the screen 156 and the input device 158 are integrated together as a touchscreen interface.

The group health monitoring device 110 may be or include a workstation, such as a computer workstation, located within an area, such as within a galley of the internal cabin 102 of the vehicle 104 (shown in FIG. 1). In at least one other embodiment, the group health monitoring device 110 may be a handheld device, such as a smart phone or tablet, carried by an attendant who is tasked with taking care of the individuals associated with the personal health assessment devices 106 (shown in FIG. 1).

Referring to FIGS. 1 and 11, in operation, the group health monitoring device 110 receives the health signals 112 including the health data for the individuals associated with the personal health assessment devices 106. The group monitoring control unit 154 displays the health status, including the health data, for the individuals on the screen 156. In at least one embodiment, the group monitoring control unit 154 shows the health status for all of the individuals associated with the personal health assessment device 106 on the screen 156. In such an embodiment, individuals in need of attention due to aspects of health data may be noted with prominent and noticeable indicia, such as color coding (for example, red indicating an individual in need of immediate attention, yellow indicating those in need of attention after those needing immediate attention are accommodated by the attendant, and green indicating those not in need of attention, or who have merely requested casual drink or food service). In at least one other embodiment, the group monitoring control unit 154 may only show the health status for those individuals needing attention due to health data. As such, the group health monitoring device 110 allows an attendant to quickly and easily discern which individuals need assistance, service, or other such attention. Further, the group health monitoring device 110 allows the attendant to determine which individuals should be attended to before others, such as due to a severity of a health concern. For example, a dehydrated individual is to be attended to before a hydrated individual who has requested a beverage. The levels of severity of health data may be determined by the health assessment control unit 128 (such as shown in FIGS. 2 and 3), such as via comparison to predetermined and stored thresholds and criteria.

FIG. 12 illustrates a front view of the user interface 152 of the group health monitoring device 110, according to an embodiment of the present disclosure. The group health monitoring device 110 may show locations of seats 108 (shown in FIG. 1) of individuals that need service, assistance, or other such attention (instead of showing all of the locations the individuals, even if service or assistance is not needed).

Referring to FIGS. 1, 11 and 12, the group monitoring control unit 154 may show a seat location 500, a health status 502 at the seat location, and a checked indication 504 for the seat location. The personal health assessment devices 106 output the health signals 112 (which are received by the group health monitoring devices 110) including the health data indicative of the health status of the individuals associated with the personal health assessment devices 106. Instead of indicating a passenger name, only a seat location may be shown in the seat location 500 shown on the screen 156. That is, the health data may not include personal identification information, such as names of the individuals.

As shown in the example of FIG. 12, the individual seated at 4A has a low hydration level, and has not been checked by an attendant yet. The individual seated at 26C is mildly fatigued, but has been checked by an attendant, as indicated by the checked indicia 510. The individual at 30B has an elevated heart rate and has not been checked by an attendant yet. The individual at 33E has an extremely low hydration level, has not been checked on yet, and should be checked before anyone else, as noted by the alert indicia 512, which may be a graphic, color code, flashing light, or the like. Various levels of alert indicia 512 may be used with respect to the locations 500 on the screen to allow the attendants to prioritize attention based on degrees of need. For example, an individual who is dehydrated should be attended to before an individual who is mildly fatigued or who is hydrated and has requested a beverage.

In general, after an attendant checks on an individual in need of service, the attendant may input the checked indicia 510, such as via a touch on the appropriate location on the screen 156. In doing so, other attendants are notified that there is no further need to check on the individual at 26C. As such, the attendants are able to quickly and easily determine who should be attended to and not contact individuals who are no longer in need of attention.

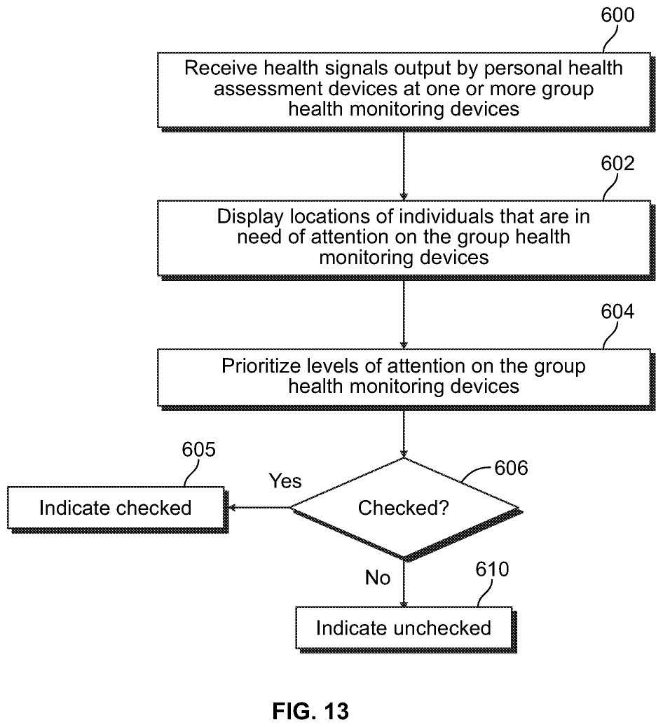

FIG. 13 illustrates a flow chart of a method of displaying health status of individuals associated with personal health assessment devices 106, according to an embodiment of the present disclosure. Referring to FIGS. 1 and 11-13, in at least one embodiment, the group monitoring control unit 154 is configured to operate according to the method shown in FIG. 13.

At 600, health signals 112 including health data indicative of health status of the individuals associated with the personal health assessment devices 106 are received by the group health monitoring devices 110. At 602, the group health monitoring devices 110 display locations of individuals that are in need of attention from the attendants on the screens 156. At 604, the levels of attention are prioritized depending on a severity of the health status. At 606, it is determined whether individuals have been checked by the attendants. If so, the individuals are noted as checked in 608. If not, the individuals are not noted as being checked yet at 610.

Certain embodiments of the present disclosure provide a health monitoring method configured to monitor health of one or more individuals within the internal cabin 102 of a vehicle 104. The health monitoring method includes associating one or more group health monitoring devices 110 with one or more attendants who are responsible for taking care of the individual(s), and receiving, by the group health monitoring devices 110, the health signals 112 including health data of the individual(s) within the internal cabin 102 from the personal health assessment devices 106.

In at least one embodiment, the health monitoring method includes receiving, by the group monitoring control unit 154 of the group health monitoring device 110, the health signals 112 from the personal health assessment device(s) 106. The health monitoring method may also include displaying, on the screen 156 of the user interface 152 of the group health monitoring device 110, at least a portion of the health data. For example, the health data of individuals in need of assistance may be displayed, instead of the health data of all of the individuals within the internal cabin 102. The displaying may include showing the seat location 500, the health status 502 at the seat location 500, and the checked indication 504 for the seat location 500. Further, the health monitoring method may include indicating prioritized attention on the screen by the alert indicia 512.

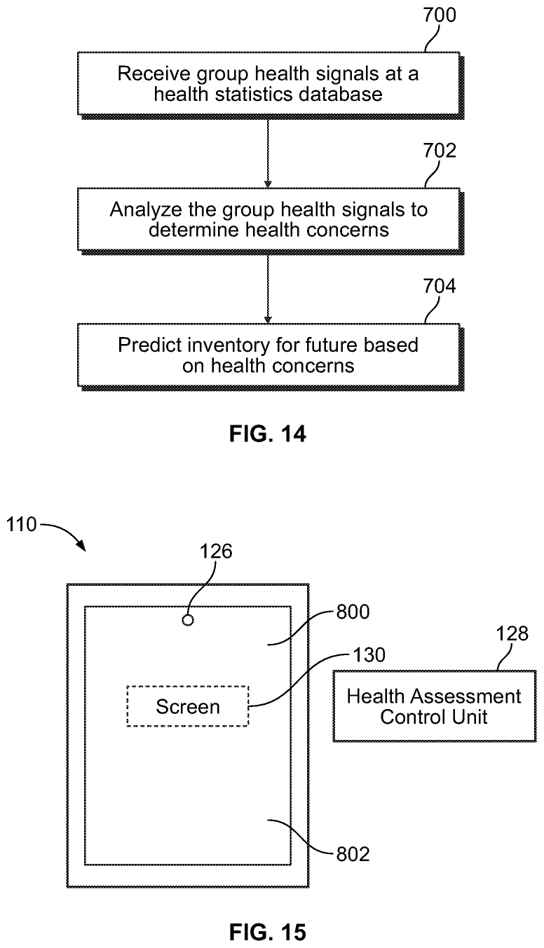

FIG. 14 illustrates a flow chart of a method of predicting inventory for future routes (such as flights) of vehicles, according to an embodiment of the present disclosure. Referring to FIGS. 1 and 14, in at least one embodiment, the inventory prediction control unit 114 operates according to the flow chart shown and described with respect to FIG. 14.

As explained above, the group health monitoring devices 110 receive and analyze health signals 112 including the health data indicative of the health status of individuals associated with the personal health assessment devices 106. The group health monitoring devices 110 may receive and analyze such health signals 112 during a route of the vehicle 104, such as a flight of a commercial aircraft. After the completion of the route, the group health monitoring devices 110 and/or the personal health assessment devices 106 may delete the health data.

In at least one other embodiment, the group health monitoring devices 110 may compile the health signals 112 for the route (for example, the flight) as group health data. At 700, a group health signal 118 that includes the group health data is transmitted or otherwise output to the health statistics database 116, which receives the group health signals 118 from at least one of the group health monitoring devices 110. The group health data of the group health signals 118 may be associated with the route and date of the route, for example.

At 702, group health data stored within the health statistics database is analyzed by the inventory prediction control unit 114 to determine health concerns during the route. The health concerns include instances of dehydration, fatigue, elevated vital signs, and the like during the route. At 704, the inventory prediction control unit 114 then predicts inventory (such as bottles of water, blankets, pillows, juices, food items, and the like) for future routes based on the instances of health concerns during the previous route.

In at least one embodiment, the health statistics database 116 stores group health signals 118 for numerous events over a selected time frame. For example, the health statistics database 116 stores group health signals 118 for flights of one or more aircraft for a week, month, year, or longer. In at least one embodiment, the group health signals 118 are associated with particular flights, dates of flights, and the like. As such, the inventory prediction control unit 114 is able to predict inventory for various flights at different times of year. For example, the inventory prediction control unit 114 analyzes the group health data as received by the group health signals 118 for flights at different times of year to determine an inventory prediction that may differ due to the different times of year.

Certain embodiments of the present disclosure provide a health monitoring method configured to monitor health of one or more individuals within the internal cabin 102 of the vehicle 104. The health monitoring method includes storing the group health data in the health statistics database 116, communicatively coupling the inventory prediction control unit 114 with the health statistics database 116, analyzing, by the inventory prediction control unit 114, the group health data stored in the health statistics database 116, and predicting, via the analyzing, future inventory for the vehicle 104.

The health monitoring method may include analyzing, by the inventory prediction control unit 114 in communication with the health statistics database 116, group health data stored in the health statistics database 116 to predict future inventory for the vehicle. Further, the health monitoring method may include receiving, by the health statistics database 116, the group health signal 118 that includes the group health data from the group health monitoring devices 110.

The health monitoring method may also include storing, in the health statistics database 116, the group health data for one or more routes (for example, flights) of the vehicle 104 or one or more routes of at least one other vehicle (for example, a different vehicle 104). The health monitoring method may also include associating the group health data with the route(s) and dates of the route(s).

FIG. 15 illustrates a schematic diagram of a personal health assessment device 106, according to an embodiment of the present disclosure. In this embodiment, the personal health assessment device 106 includes a mirror 800 having a reflective surface 802 that reflects an image of an individual. The reflective surface 802 allows for an image to be reflected, but also for the imaging device 126 to capture the image through the reflective surface 802, and the screen 130 to show graphics (such as pictures, videos, etc.), text, and the like on the reflective surface 802. The personal health assessment device 106 may operate as described above.

Notably, when an individual looks into the mirror 800, the health assessment control unit 128 may obtain health data via images of the individual captured by the imaging device 126, as described above. The health assessment control unit 128 may then show health data for the individual on the reflective surface 802 via the screen 130, as well as provide suggestions to alleviate potential health concerns. For example, the health assessment control unit 128 may display a message on the reflective surface 802, via the screen 130, that the individual is mildly dehydrated and may consider drinking a glass of water.

In at least one embodiment, a health monitoring method includes capturing an image, by the imaging device 126 of the personal health assessment device 106, of an individual through the reflective surface 802 of the mirror 800, and showing graphics and text on the reflective surface 802.

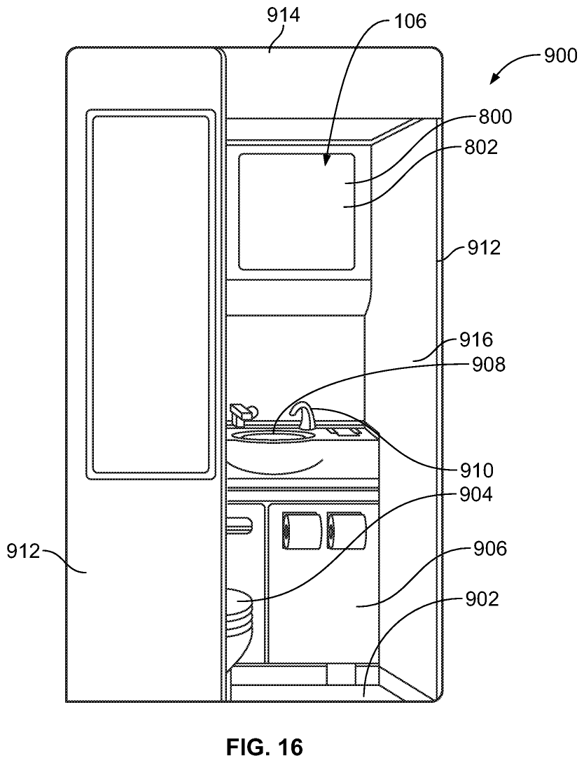

FIG. 16 illustrates a perspective internal view of a lavatory 900, according to an example of the present disclosure. The lavatory 900 may be onboard an aircraft, for example. Optionally, the lavatory 900 may be onboard various other vehicles, such as automobiles, buses, locomotives and train cars, ships, watercraft, and the like. In other examples, the lavatory 900 may be within a fixed structure, such as a commercial or residential building, or outbuilding.

The lavatory 900 includes a floor 902 that supports a toilet 904, cabinets 906, a sink 908, and a faucet 910. The lavatory 900 is enclosed by walls 912 that connect to the floor 902 and a ceiling 914. A framed opening 916 is formed through one of the walls 912, and is configured to retain a door (not shown), which is moveable between open and closed positions.

Referring to FIGS. 15 and 16, in at least one embodiment, the personal health assessment device 106 is positioned above the sink 908. An individual washing hands in the sink 908 is able to see a reflection in the mirror 800. At the same time, the personal health assessment device 106 may obtain health data from the individual and display the health data and messages on the reflective surface 802, thereby providing the individual with a quick and easy health assessment. In embodiments in which the personal health assessment device 106 is integrated into a lavatory 106, such as onboard an aircraft, the personal health assessment device 106 does not include an imaging device.

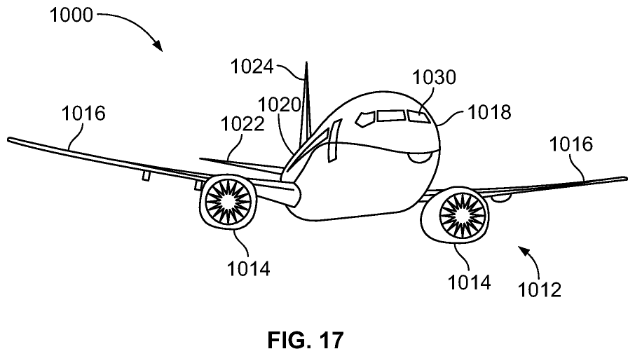

FIG. 17 illustrates a front perspective view of an aircraft 1000, according to an exemplary embodiment of the present disclosure. The aircraft 1000 is an example of the vehicle 104 shown in FIG. 1. The aircraft 1000 includes a propulsion system 1012 that may include two turbofan engines 1014, for example. Optionally, the propulsion system 1012 may include more engines 1014 than shown. The engines 1014 are carried by wings 1016 of the aircraft 1000. In other embodiments, the engines 1014 may be carried by a fuselage 1018 and/or an empennage 1020. The empennage 1020 may also support horizontal stabilizers 1022 and a vertical stabilizer 1024. The fuselage 1018 of the aircraft 1000 defines an internal cabin (such as the internal cabin 102 shown in FIG. 1), which may include a cockpit 1030.

The aircraft 1000 may be sized, shaped, and configured other than shown in FIG. 17. For example, the aircraft 1000 may be a non-fixed wing aircraft, such as a helicopter. As another example, the aircraft 1000 may be an unmanned aerial vehicle (UAV).

As used herein, the term “control unit,” “central processing unit,” “unit,” “CPU,” “computer,” “module,” or the like may include any processor-based or microprocessor-based system including systems using microcontrollers, reduced instruction set computers (RISC), application specific integrated circuits (ASICs), logic circuits, and any other circuit or processor including hardware, software, or a combination thereof capable of executing the functions described herein. Such are exemplary only, and are thus not intended to limit in any way the definition and/or meaning of such terms. For example, the health assessment control unit 128, the group monitoring control unit 154, the inventory prediction control unit 114, and any modules thereof may be or include one or more processors that are configured to control operation thereof, as described herein.

The health assessment control unit 128, the group monitoring control unit 154, the inventory prediction control unit 114, and any modules thereof are configured to execute a set of instructions that are stored in one or more data storage units or elements (such as one or more memories), in order to process data. For example, the health assessment control unit 128, the group monitoring control unit 154, the inventory prediction control unit 114, and any modules thereof may include or be coupled to one or more memories. The data storage units may also store data or other information as desired or needed. The data storage units may be in the form of an information source or a physical memory element within a processing machine.

The set of instructions may include various commands that instruct the health assessment control unit 128, the group monitoring control unit 154, the inventory prediction control unit 114, and any modules thereof as processing machines to perform specific operations such as the methods and processes of the various embodiments of the subject matter described herein. The set of instructions may be in the form of a software program. The software may be in various forms such as system software or application software. Further, the software may be in the form of a collection of separate programs, a program subset within a larger program or a portion of a program. The software may also include modular programming in the form of object-oriented programming. The processing of input data by the processing machine may be in response to user commands, or in response to results of previous processing, or in response to a request made by another processing machine.

The diagrams of embodiments herein may illustrate one or more control or processing units, such as the health assessment control unit 128, the group monitoring control unit 154, the inventory prediction control unit 114, and any modules thereof. It is to be understood that the processing or control units may represent circuits, circuitry, or portions thereof that may be implemented as hardware with associated instructions (e.g., software stored on a tangible and non-transitory computer readable storage medium, such as a computer hard drive, ROM, RAM, or the like) that perform the operations described herein. The hardware may include state machine circuitry hardwired to perform the functions described herein. Optionally, the hardware may include electronic circuits that include and/or are connected to one or more logic-based devices, such as microprocessors, processors, controllers, or the like. Optionally, the health assessment control unit 128, the group monitoring control unit 154, the inventory prediction control unit 114, and any modules thereof may represent processing circuitry such as one or more of a field programmable gate array (FPGA), application specific integrated circuit (ASIC), microprocessor(s), and/or the like. The circuits in various embodiments may be configured to execute one or more algorithms to perform functions described herein. The one or more algorithms may include aspects of embodiments disclosed herein, whether or not expressly identified in a flowchart or a method.

As used herein, the terms “software” and “firmware” are interchangeable, and include any computer program stored in a data storage unit (for example, one or more memories) for execution by a computer, including RAM memory, ROM memory, EPROM memory, EEPROM memory, and non-volatile RAM (NVRAM) memory. The above data storage unit types are exemplary only, and are thus not limiting as to the types of memory usable for storage of a computer program.

Referring to FIGS. 1-17, embodiments of the present disclosure provide systems and methods that allow large amounts of data to be quickly and efficiently analyzed by a computing device. For example, numerous passengers may be onboard a flight. Further, numerous flights may be flown over a particular time frame. As such, large amounts of health data are being tracked and analyzed. The vast amounts of data are efficiently organized and/or analyzed by the health assessment control unit 128, the group monitoring control unit 154, the inventory prediction control unit 114, and any modules thereof, as described herein. The health assessment control unit 128, the group monitoring control unit 154, the inventory prediction control unit 114, and any modules thereof analyze the data, as described herein. A human being would be incapable of efficiently analyzing such vast amounts of data in such a short time. Attendants need not check in on every passenger to determine health status of the passengers. Instead, embodiments of the present disclosure automatically detect health status of the passengers, and allow the attendants to focus their attention on passengers who need attention. As such, embodiments of the present disclosure provide increased and efficient functionality with respect to prior computing systems, and vastly superior performance in relation to a human being analyzing the vast amounts of data. In short, embodiments of the present disclosure provide systems and methods that analyze thousands, if not millions, of calculations and computations that a human being is incapable of efficiently, effectively and accurately managing.

As described herein, embodiments of the present disclosure provide systems and methods for attending to needs of a group of individuals, such as onboard an aircraft. Further, embodiments of the present disclosure provide systems and methods for attending to the needs of the group of individuals that need not rely on the individuals themselves to expressly contact the attendants for service and assistance. Moreover, embodiments of the present disclosure provide systems and methods for attending to a group of individuals onboard an aircraft that does not require a passenger to engage a physical call button. Also, embodiments of the present disclosure provide systems and methods for analyzing health data from previous routes (for example flights) to predict inventory needs for future routes.

While various spatial and directional terms, such as top, bottom, lower, mid, lateral, horizontal, vertical, front and the like may be used to describe embodiments of the present disclosure, it is understood that such terms are merely used with respect to the orientations shown in the drawings. The orientations may be inverted, rotated, or otherwise changed, such that an upper portion is a lower portion, and vice versa, horizontal becomes vertical, and the like.

As used herein, a structure, limitation, or element that is “configured to” perform a task or operation is particularly structurally formed, constructed, or adapted in a manner corresponding to the task or operation. For purposes of clarity and the avoidance of doubt, an object that is merely capable of being modified to perform the task or operation is not “configured to” perform the task or operation as used herein.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the various embodiments of the disclosure without departing from their scope. While the dimensions and types of materials described herein are intended to define the parameters of the various embodiments of the disclosure, the embodiments are by no means limiting and are exemplary embodiments. Many other embodiments will be apparent to those of skill in the art upon reviewing the above description. The scope of the various embodiments of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112(f), unless and until such claim limitations expressly use the phrase “means for” followed by a statement of function void of further structure.

This written description uses examples to disclose the various embodiments of the disclosure, including the best mode, and also to enable any person skilled in the art to practice the various embodiments of the disclosure, including making and using any devices or systems and performing any incorporated methods. The patentable scope of the various embodiments of the disclosure is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal language of the claims.

1. A health monitoring system configured to monitor health of one or more individuals within an internal cabin of a vehicle, the health monitoring system comprising:
one or more personal health assessment devices associated with the one or more individuals, wherein the one or more personal health assessment devices obtain health data from the one or more individuals and output health signals including the health data to one or more group health monitoring devices associated with one or more attendants who are responsible for taking care of the one or more individuals.

2. The health monitoring system of claim 1, wherein the one or more personal health assessment devices comprise a plurality of personal health assessment devices associated with a plurality of the individuals, wherein each of the plurality of personal health assessment devices is associated with a respective one of the plurality of the individuals.

3. The health monitoring system of claim 1, wherein the one or more personal health assessment devices are associated with one or more seats within the internal cabin.

4. The health monitoring system of claim 1, wherein the one or more personal health assessment devices comprise a health assessment control unit that obtains the health data.

5. The health monitoring system of claim 4, wherein the one or more personal health assessment devices further comprises a user interface in communication with the health assessment control unit, wherein the health data is input into the health assessment control unit via an input device of the user interface.

6. The health monitoring system of claim 5, wherein the user interface comprises a virtual call button.

7. The health monitoring system of claim 5, wherein the one or more personal health assessment devices further comprises an imaging device in communication with the health assessment control unit, wherein the imaging device captures image data of the one or more individuals associated with the one or more personal health assessment devices, wherein the health assessment control unit analyzes the image data to determine the health data.

8. The health monitoring system of claim 5, wherein the one or more personal health assessment devices further comprises one or more health monitoring devices in communication with the health assessment control unit, wherein the one or more health monitoring devices obtains the health data.

9. The health monitoring system of claim 5, wherein the health assessment control unit comprises an interaction module that displays messages on a screen and allows the one or more individuals to input the health data.

10. The health monitoring system of claim 5, wherein the health assessment control unit comprises:
a hydration assessment module that analyzes images of the one or more individuals to determine a hydration level of the one or more individuals;
a fatigue assessment module that analyzes the images to determine a fatigue level of the one or more individuals;
a heart rate assessment module that analyzes the images to determine a heart rate of the one or more individuals; and
an oxygen level assessment module that analyzes the images to determine a blood oxygen level of the one or more individuals.

11. The health monitoring system of claim 5, wherein the health data relates to one or more of hydration level, fatigue level, heart rate, blood pressure, or blood oxygen level.

12. The health monitoring system of claim 1, wherein the one or more personal health assessment devices comprises:
a mirror having a reflective surface;
an imaging device that captures an image of the one or more individuals through the reflective surface; and
a screen that shows graphics and text on the reflective surface.

13. A health monitoring method configured to monitor health of one or more individuals within an internal cabin of a vehicle, the health monitoring method comprising:
associating one or more personal health assessment devices with the one or more individuals;
obtaining, by the one or more personal health assessment devices, health data from the one or more individuals; and
outputting, by the one or more personal health assessment devices, health signals including the health data to one or more group health monitoring devices associated with one or more attendants who are responsible for taking care of the one or more individuals.

14. The health monitoring method of claim 13, wherein the associating comprises associating a plurality of personal health assessment devices with a plurality of the individuals, wherein each of the plurality of personal health assessment devices is associated with a respective one of the plurality of the individuals.

15. The health monitoring method of claim 13, wherein the associating comprises associating the one or more personal health assessment devices with one or more seats within the internal cabin.

16. The health monitoring method of claim 13, wherein the obtaining comprises obtaining the health data with a health assessment control unit of the one or more personal health assessment devices.

17. The health monitoring method of claim 16, wherein the obtaining comprises inputting the health data into the health assessment control unit via an input device of a user interface.

18. The health monitoring method of claim 16, further comprising providing a virtual call button on the user interface.

19. The health monitoring method of claim 16, wherein the obtaining comprises:
capturing image data of the one or more individuals by an imaging device; and
analyzing the image data by the health assessment control unit to determine the health data.

20. The health monitoring method of claim 16, wherein the obtaining comprises obtaining the health data by one or more health monitoring devices in communication with the health assessment control unit.

21. The health monitoring method of claim 13, further comprising:
capturing an image, by an imaging device of the one or more personal health assessment devices, of the one or more individuals through a reflective surface of a mirror; and
showing graphics and text on the reflective surface.
